(19) United States
(12) Patent Application Publication
(10) Pub. No.: US 2026/0256731 A1
(43) Pub. Date: Sep. 3, 2026

(54) RETINAL VASODILATOR AND PHARMACEUTICAL COMPOSITION

(71) Applicant: TEIKYO UNIVERSITY, Tokyo (JP)

(72) Inventors: Yoshiki Miyata, Tokyo (JP); Taiji Nagaoka, Tokyo (JP); Masahisa Watanabe, Tokyo (JP)

(21) Appl. No.: 18/846,479

(22) PCT Filed: Mar. 10, 2023
(86) PCT No.: PCT/JP2023/009296

§ 371 Date: Sep. 12, 2024

(30) Foreign Application Priority Data
Mar. 15, 2022 (JP) 2022-040830

(51) Int. Cl. A61K 31/352; A61P 9/08; A61P 27/02

(52) U.S. Cl. CPC A61K 31/352; A61P 9/08; A61P 27/02

(57) ABSTRACT

A retinal vasodilator containing nobiletin is provided. A pharmaceutical composition for treating or preventing retinal circulatory disturbance containing the above retinal vasodilator and a pharmaceutically acceptable carrier is also provided.

### TECHNICAL FIELD

The present invention relates to a retinal vasodilator and a pharmaceutical composition.

Priority is claimed on Japanese Patent Application No. 2022-040830, filed on Mar. 15, 2022, the content of which is incorporated herein by reference.

### BACKGROUND ART

Retinal circulatory disturbance is caused earlier than discovery of an obvious finding of retinal-related disease, and is one of the causes of onset of various retinal-related diseases. In the case where retinal circulatory disturbance occurs, blood vessel neogenesis occurs in order to compensate for blood flow insufficiency. The newly formed blood vessel formed in this manner has a fragile blood vessel wall, and is likely to cause bleeding or blood component leakage. Therefore, edema or the like occurs, and symptoms such as a decrease in visual acuity, visual field constriction, and visual field defect are caused. In the case where the symptoms have progressed, laser photocoagulation, vitreous body surgery, or the like is used, but these treatment methods have high invasiveness. For macular edema, intravitreal injection of a VEGF inhibitor is performed, but the cost of medication thereof is high. In addition, since intravitreal injection is invasive, repeat treatment is considered to be difficult.

It is considered that, in the case where retinal circulatory disturbance can be improved at an earlier stage before the occurrence of edema or the like, the expression of the symptoms of retinal-related disease can be suppressed. Retinal circulatory disturbance is improved by using a retinal circulation improvement medication, but it is difficult for an existing medication to reach an effective concentration for dilating retinal blood vessels in a living body.

Nobiletin is a kind of flavonoid contained in citrus fruits such as citrus depressa. It has been reported that nobiletin has a vasodilating action on rat aorta (Non-Patent Document 1) and rat mesenteric artery (Non-Patent Document 2) that have been pre-contracted with phenylephrine. In Non-Patent Document 1, it is considered that nobiletin expands the rat aorta in a vascular endothelium-independent manner. In Non-Patent Document 2, it is considered that nobiletin expands the rat mesenteric artery in a vascular endothelium-dependent manner.

Blood vessel dilation mechanisms vary depending on a tissue in which the blood vessel is present, and blood vessel dilation response to a compound also varies depending on the tissue. For example, in Non-Patent Document 2, it is reported that a vasodilator response by nobiletin did not occur in a rat pulmonary artery.

### CITATION LIST

### Non-Patent Documents

Non-Patent Document 1: Kaneda et al., Kaneda et al., Endothelium-independent vasodilator effects of nobiletin in rat aorta. J Pharmacol Sci. 2019 May; 140 (1): 48-53.
Non-Patent Document 2: Yang et al., Nobiletin Relaxes Isolated Mesenteric Arteries by Activating the Endothelial Ca2+-eNOS Pathway in Rats. J Vasc Res. 2016; 53 (5-6): 330-339.

### SUMMARY OF INVENTION

### Technical Problem

It is considered that, in the case where retinal circulatory disturbance can be improved at an early stage, the occurrence and progression of symptoms such as edema and visual impairment can be suppressed. Therefore, there is a demand for development of a retinal vasodilator having few side effects and a high vasodilating effect.

Therefore, an object of the present invention is to provide a novel retinal vasodilator, and a pharmaceutical composition containing the retinal vasodilator.

### Solution to Problem

The present invention includes the following aspects.

[1] A retinal vasodilator containing:
nobiletin.

[2] A pharmaceutical composition for treating or preventing retinal circulatory disturbance, the pharmaceutical composition containing:
the retinal vasodilator according to [1]; and
a pharmaceutically acceptable carrier.

### Advantageous Effects of Invention

According to the present invention, a novel retinal vasodilator, and a pharmaceutical composition containing the retinal vasodilator are provided.

### BRIEF DESCRIPTION OF DRAWINGS

FIG. 1 represents results of evaluating the vasodilating action of nobiletin on an extracted porcine retinal artery.

FIG. 2 represents results of evaluating the vasodilating action of nobiletin on an extracted porcine retinal artery; in which a test of n=5 is performed twice.

FIG. 3 represents results of evaluating the vasodilating action of nobiletin using an extracted porcine retinal artery with denudation of the vascular endothelium.

FIG. 4 represents results of evaluating the vasodilating action of nobiletin using an extracted porcine retinal artery treated with ODQ, which is a guanylate cyclase inhibitor.

### DESCRIPTION OF EMBODIMENTS

### [Retinal Vasodilator]

A first aspect of the present invention relates to a retinal vasodilator containing nobiletin.

Nobiletin (2-(3,4-Dimethoxyphenyl)-5,6,7,8-tetramethoxychromen-4-one) is a kind of polymethoxyflavonoid having a flavone skeleton. Nobiletin is represented by the following chemical formula.

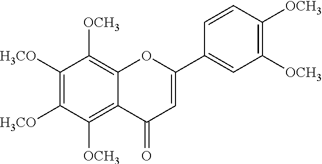 [SMILES: COc1ccc(-c2cc(=O)c3c(OC)c(OC)c(OC)c(OC)c3o2)cc1OC]

Nobiletin is particularly contained in a large amount in the peel of citrus fruits such as citrus depressa, citrus reticulata, citrus sphaerocarpa, and citrus tachibana. Nobiletin may be purified from the peel of these citrus fruits by a known method, or a commercially available product thereof may be used.

“Retinal blood vessel” means a blood vessel present in the retina. The retinal blood vessel may be a retinal artery or a retinal vein. The retinal vasodilator according to the present aspect acts on the retinal blood vessel to vasodilate the retinal blood vessel. The retinal vasodilator according to the present aspect contains nobiletin as an active ingredient, which has a strong vasodilating action on the retina. Therefore, the retinal vasodilator according to the present aspect exhibits a strong vasodilating action on the retinal blood vessel.

The retinal vasodilator according to the present aspect may contain an optional component in addition to nobiletin. The optional component is not particularly limited. As the optional component, various additives used in manufacturing of pharmaceuticals, cosmetics, foods, and the like can be used without particular limitation. Regarding the optional component, for example, a pharmaceutically acceptable carrier is an exemplary example. The “pharmaceutically acceptable carrier” means a carrier which does not inhibit physiological activity of an active ingredient and does not exhibit substantial toxicity to a target of administration of the active ingredient. The phrase “does not exhibit substantial toxicity” means that, at the dosage in which a component is usually used, the component does not exhibit toxicity in the target of administration. In the retinal vasodilator according to the present aspect, the pharmaceutically acceptable carrier is a carrier which does not inhibit the retinal vasodilating action of nobiletin and does not exhibit substantial toxicity to the target of administration thereof. The pharmaceutically acceptable carrier is typically considered to be an inactive ingredient. The pharmaceutically acceptable carrier may include any known pharmaceutically acceptable components. As the pharmaceutically acceptable carrier, for example, a solvent, a diluent, a vehicle, an excipient, a flow promoter, a binding agent, a granulating agent, a dispersing agent, a suspending agent, a wetting agent, a lubricant, a disintegrating agent, a solubilizing agent, a stabilizer, an emulsifier, a filling agent, a preservative (for example, an antioxidant), a chelating agent, a surfactant, a thickener, a gelling agent, a buffer, water, an inorganic acid, an organic acid, an inorganic base, an organic base, an alcohol, an inorganic salt, a polymer additive, a metal-containing compound, a polymer carboxylic acid, and the like are exemplary examples, but the pharmaceutically acceptable carrier is not limited thereto. As the pharmaceutically acceptable carrier, one type may be used alone, or two or more types may be used in combination.

Dosage forms of the retinal vasodilator according to the present aspect are not particularly limited, and any dosage form may be used. As the dosage forms, for example, a tablet, a granule, a powder, a pill, a liquid, an emulsion, a capsule, and the like are exemplary examples. The retinal vasodilator of these dosage forms can be manufactured by adding an appropriate additive to nobiletin according to the dosage form and formulating the mixture according to a standard method.

The retinal vasodilator according to the present aspect may be administered to a target requiring retinal vasodilating itself, or may be used by being formulated in a pharmaceutical product, a cosmetic product, a food, a feed, or the like.

As the target requiring retinal vasodilating, for example, a target in which a retinal circulatory disturbance such as retinal blood vessel stenosis and retinal blood vessel occlusion has occurred due to various factors, or a target having a high risk of occurrence of retinal circulatory disturbance are exemplary examples. The retinal vasodilator according to the present aspect may be administered to a target in which retinal circulatory disturbance has occurred in order to improve these symptoms, or may be administered to a target at a high risk of occurrence of retinal circulatory disturbance in order to prevent these symptoms. Retinal circulatory disturbance can occur due to, for example, arteriosclerosis, hypertension, diabetes, heart disease, aging, and the like. Therefore, as the target having a high risk of retinal circulatory disturbance, for example, a target having a disease or a symptom, such as arteriosclerosis, hypertension, diabetes, and heart disease; an elderly person; and the like are exemplary examples.

Applicable target of the retinal vasodilator according to the present aspect is not particularly limited as long as it is an animal having a retina. Examples of applicable targets include, for example, humans and mammals other than humans. Mammals other than human are not particularly limited, but include primates (monkey, chimpanzee, gorilla, and the like), rodents (mouse, hamster, rat, and the like), rabbit, dog, cat, cow, goat, sheep, horse, and the like.

### [Pharmaceutical Composition]

A second aspect of the present invention relates to a pharmaceutical composition for treating or preventing retinal circulatory disturbance, the pharmaceutical composition containing the retinal vasodilator according to the first aspect, and a pharmaceutically acceptable carrier.

The pharmaceutical composition according to the present aspect contains nobiletin as an active ingredient. Since nobiletin has a strong vasodilating action on retinal blood vessels, the pharmaceutical composition according to the present aspect can be used for treating or preventing retinal circulatory disturbance such as retinal blood vessel stenosis and retinal blood vessel occlusion.

“Retinal circulation” means blood flow in the retina. “Retinal circulatory disturbance” means disturbed blood flow in the retina. Retinal circulatory disturbance is a disorder caused by occlusion or stenosis of retinal blood vessels. In the case where retinal circulatory disturbance occurs due to occlusion or stenosis of retinal blood vessels, supply of oxygen and nutrients to retinal cells and an optic nerve is hindered. Retinal circulatory disturbance is one of the causes of various retinal-related diseases. As such retinal-related diseases, for example, diabetic retinopathy, retinal vein occlusion, retinal artery occlusion, age-related macular degeneration, and central serous chorioretinopathy are exemplary examples, but such retinal-related diseases are not limited thereto.

Diabetic retinopathy is a disease in which chronic hyperglycemia due to diabetes causes a disorder in the retina. In the case of suffering from diabetes, a blood sugar level is increased, and retinal blood vessels are impaired by the sugar, which makes it easier for the blood vessels to be narrowed or blocked. In the case where the retinal blood vessels are impaired, an ischemic region is gradually expanded, and new blood vessels are formed to compensate for the blood flow deficiency. Since the blood vessel wall of new blood vessels is fragile, bleeding and leakage of blood components are likely to occur, which causes macular edema. In the case where the symptoms progress, visual acuity may be decreased, and blindness may occur in some cases due to proliferative change and the complication of the traction retinal detachment or the like. In diabetic retinopathy, retinal circulatory disturbance occurs from an early stage, and it is considered that progression of symptoms can be suppressed by improving retinal circulatory disturbance.

The pharmaceutical composition according to the present aspect can be administered to a subject suffering from diabetic retinopathy in order to improve retinal circulatory disturbance and inhibit the progression of diabetic retinopathy. In diabetic retinopathy, retinal circulatory disturbance occurs from an early stage, and is one of the causes of subsequent progression of symptoms. Therefore, the pharmaceutical composition according to the present aspect can be particularly suitably applied to a target at an early stage of diabetic retinopathy. The target to be administered with the pharmaceutical composition according to the present aspect may suffer from diabetes and may not have developed diabetic retinopathy, and may be administered preventively. For a patient with diabetes, by improving or preventing retinal circulatory disturbance, the onset of diabetic retinopathy can be suppressed.

Retinal vein occlusion is a disease in which the retinal vein is occluded. In the case where the retinal vein is occluded, bleeding, macular edema, and the like occur. Retinal vein occlusion is classified into central retinal vein occlusion, branch retinal vein occlusion, and the like, depending on the site of vein occlusion. The pharmaceutical composition according to the present aspect can be applied to either central retinal vein occlusion or branch retinal vein occlusion.

The pharmaceutical composition according to the present aspect can be administered to a subject who has developed retinal vein occlusion in order to dilate the retinal vein and improve occlusion of the retinal vein. Retinal vein occlusion is likely to occur in the case where a symptom such as hypertension and arteriosclerosis is exhibited. Therefore, the pharmaceutical composition according to the present aspect may be administered preventively to a target having hypertension, arteriosclerosis, and/or the like. In a target group of these high-risk groups, it is possible to suppress the onset of retinal vein occlusion by improving or preventing retinal circulatory disturbance.

Retinal artery occlusion is a disease in which the retinal artery is occluded. In the case where the retinal artery is occluded, supply of oxygen and nutrients to retinal cells is interrupted, and the retinal cells die. Retinal artery occlusion is classified into central retinal artery occlusion, branch retinal artery occlusion, and the like, depending on the site of vein occlusion. The pharmaceutical composition according to the present aspect can be applied to both central retinal artery occlusion and branch retinal arterial occlusion.

The pharmaceutical composition according to the present aspect can be administered to a subject who has developed retinal artery occlusion in order to dilate the retinal artery and improve occlusion of the retinal artery. Retinal artery occlusion is likely to occur in the case where a symptom such as hypertension and arteriosclerosis is exhibited. Therefore, the pharmaceutical composition according to the present aspect may be administered preventively to a target having hypertension, arteriosclerosis, and/or the like. In a target group of these high-risk groups, it is possible to suppress the onset of retinal artery occlusion by improving or preventing retinal circulatory disturbance.

Age-related macular degeneration is a disease in which the macula is degenerated due to changes with aging. Age-related macular degeneration is classified into an exudative type accompanied by choroidal neovascularization and an atrophic type in which the tissue of the retina gradually atrophies. It is considered that retinal circulatory disturbance is involved in the development of choroidal neovascularization or atrophy of the retinal tissue. The pharmaceutical composition according to the present aspect can be applied to either exudative age-related macular degeneration or atrophic age-related macular degeneration.

The pharmaceutical composition according to the present aspect can be administered to a subject suffering from the age-related macular degeneration in order to improve retinal circulatory disturbance by dilating retinal blood vessels and inhibit the progression of age-related macular degeneration. Age-related macular degeneration is likely to occur with age, and thus is likely to occur in elderly person. Therefore, the pharmaceutical composition according to the present aspect may be administered to an elderly person preventively. In an elderly person, by improving or preventing retinal circulatory disturbance, the onset of age-related macular degeneration can be suppressed.

Central serous chorioretinopathy is a disease in which fluid accumulates in the macula and partial retinal detachment occurs. As a cause of the accumulation of fluid in the macula, leakage of serum in the choroid to the retina side due to a decrease in barrier function of the retinal pigment epithelium is an exemplary example. It is considered that retinal circulatory disturbance is involved in the decrease in barrier function of the retinal pigment epithelium. Therefore, the pharmaceutical composition according to the present aspect can be applied to central serous chorioretinopathy.

The pharmaceutical composition according to the present aspect can be administered to a subject suffering from central serous chorioretinopathy in order to improve retinal circulatory disturbance by dilating retinal blood vessels and inhibit the progression of central serous chorioretinopathy. Central serous chorioretinopathy tends to occur in men of 20 to 50 years of age, and tends to occur due to overwork, lack of sleep, stress, and the like. Therefore, the pharmaceutical composition according to the present aspect may be administered preventively to a target having a relatively high risk of developing the diseases described above. In a target group of these high-risk groups, it is possible to suppress the onset of central serous chorioretinopathy by improving or preventing retinal circulatory disturbance.

Applicable target of the pharmaceutical composition according to the present aspect is not particularly limited as long as it is to an animal having a retina. Examples of applicable target include, for example, humans and mammals other than humans are exemplary examples. Mammals other than human are not particularly limited, but include primates (monkey, chimpanzee, gorilla, and the like), rodents (mouse, hamster, rat, and the like), rabbit, dog, cat, cow, goat, sheep, horse, and the like.

The pharmaceutical composition according to the present aspect contains a pharmaceutically acceptable carrier, in addition to the retinal vasodilator according to the first aspect. The pharmaceutically acceptable carrier is not particularly limited, and a carrier usually used in pharmaceuticals can be used. As specific examples of the pharmaceutically acceptable carrier, the above-described carriers are exemplary examples. As the pharmaceutically acceptable carrier, one type may be used alone, or two or more types may be used in combination.

The pharmaceutical composition according to the present aspect may contain an optional component, in addition to the retinal vasodilator according to the first aspect and the pharmaceutically acceptable carrier. The optional component is not particularly limited, and a general pharmaceutical additive can be used without particular limitation. As the optional component, an active ingredient other than the retinal vasodilator according to the first aspect may be used. As the above-described active ingredient, preservatives, antibacterial agents, vitamins and derivatives thereof, anti-inflammatory agents, anti-inflammatory medications, blood circulation promoters, stimulants, hormones, coolants, warm sensation agents, wound healing promoters, stimulant mitigators, analgesics, cell activators, plant/animal/microorganism extracts, coolants, enzymes, nucleic acids, anti-inflammatory analgesics, antifungal agents, antihistamines, hypnotics, sedatives, antihypertensive agents, antihypertensive diuretics, antibiotics, anesthetics, antibacterial substances, antiepileptic agents, coronary vasodilators, crude medications, antipruritics, ultraviolet ray blocking agents, preservatives and bactericides, antioxidants, and the like are exemplary examples, but the active ingredient is not limited thereto. In addition to the above, as the optional component, for example, a fragrance, a colorant, a coloring agent, a dye, a pigment, a pH adjuster, a metallic soap, a taste and odor improver, a sweetener, and the like are exemplary examples, but the optional component is not limited thereto. As the optional component, one type may be used alone, or two or more types may be used in combination.

Dosage forms of the pharmaceutical composition according to the present aspect are not particularly limited, and a dosage form usually used as a pharmaceutical preparation can be appropriately adopted. As the dosage form of the pharmaceutical composition according to the present aspect, for example, dosage forms which are orally administered, such as a tablet, a coated tablet, a granule, a powder, a granule, a capsule, a liquid preparation, a suspension, and an emulsion; and dosage forms which are non-orally administered, such as eye drops, an injection, and a skin external preparation are exemplary examples. The pharmaceutical composition of these dosage forms can be formulated according to a standard method.

The pharmaceutical composition according to the present aspect is preferably an oral agent or eye drops.

In the case of an oral agent, as an additive, for example, excipients such as glucose, lactose, D-mannitol, starch, and crystalline cellulose; disintegrants or disintegration aids, such as carboxymethyl cellulose, starch, and carboxymethyl cellulose calcium; binding agents such as hydroxypropyl cellulose, hydroxypropyl methylcellulose, polyvinylpyrrolidone, and gelatin; lubricants such as magnesium stearate and talc; and bases such as hydroxypropyl methylcellulose, white sugar, polyethylene glycol, gelatin, kaolin, glycerin, purified water, and hard fat can be used.

In the case of eye drops, a dispersion medium, a thickener, a preservative, an isotonic agent, a pH adjuster, a buffer, and the like can be used in an appropriate combination.

As the dispersion medium used in eye drops, water is preferable. As the water, water of a grade usable for eye drops, such as purified water, sterilized purified water, water for injection, and distilled water for injection, can be used.

As the thickener used in eye drops, for example, cellulose-based thickeners such as methylcellulose, hydroxyethylcellulose, and hydroxypropyl methylcellulose; vinyl-based thickeners such as a carboxyvinyl polymer, a (completely or partially saponified) polyvinyl alcohol, a polyvinylpyrrolidone; and sodium alginate, sodium chondroitin sulfate, and macrogol are exemplary examples, but the thickener is not limited thereto. As the thickener, one type may be used alone, or two or more types may be used in combination.

As the preservative used in eye drops, for example, invert soaps such as benzalkonium chloride, benzethonium chloride, and chlorhexidine gluconate; parabens such as methylparaben, ethylparaben, propylparaben, and butylparaben; and alcohols such as chlorobutanol, phenylethyl alcohol, and benzyl alcohol are exemplary examples, but the preservative is not limited thereto. As the preservative, one type may be used alone, or two or more types may be used in combination.

As the isotonic agent used in eye drops, for example, sugars such as glucose; polyhydric alcohols such as propylene glycol, glycerin, mannitol, sorbitol, and xylitol; and inorganic salts such as sodium chloride and potassium chloride are exemplary examples, but the isotonic agent is not limited thereto. As the preservative, one type may be used alone, or two or more types may be used in combination.

As the pH adjuster used in eye drops, for example, dilute hydrochloric acid, triethanolamine, and the like are exemplary examples, but the pH adjuster is not limited thereto. As the pH adjuster, one type may be used alone, or two or more types may be used in combination.

As the buffer used in eye drops, for example, sodium citrate hydrate, sodium acetate hydrate, sodium hydrogen carbonate, trometamol, boric acid, borax, sodium hydrogen phosphate hydrate, and sodium dihydrogen phosphate are exemplary examples, but the buffer is not limited thereto. As the buffer, one type may be used alone, or two or more types may be used in combination.

A method for administering the pharmaceutical composition according to the present aspect is not particularly limited, and the pharmaceutical composition according to the present aspect can be administered by a method generally used as a method for administering a pharmaceutical product. The administration route may be oral or non-oral.

In the case of non-oral administration, intravenous administration, intramuscular administration, intradermal administration, subcutaneous administration, transdermal administration, and eye administration are exemplary examples; and eye administration with eye drops is preferable.

The pharmaceutical composition according to the present aspect can contain the retinal vasodilator according to the first aspect in a therapeutically effective amount. A “therapeutically effective amount” means an amount of medication effective for the treatment or prevention of a target disease. A therapeutically effective amount may vary depending on disease state, age, sex, body weight, and the like of an administration target. In the pharmaceutical composition according to the present aspect, a therapeutically effective amount of the retinal vasodilator is an amount at which nobiletin can exhibit a retinal vasodilating action. The pharmaceutical composition according to the present aspect may contain a therapeutically effective amount of nobiletin per unit dosage form. As an example, a therapeutically effective amount of the vasodilator according to the first aspect is, for example, 0.01% to 50% by mass, 0.1% to 40% by mass, 0.1% to 30% by mass, 0.1% to 20% by mass, 0.1% to 10% by mass, 0.1% to 5% by mass, or 0.1% to 3% by mass as the content of nobiletin in the pharmaceutical composition.

The pharmaceutical composition according to the present aspect can be administered in a therapeutically effective amount of the retinal vasodilator according to the first aspect. In the retinal vasodilator according to the present aspect, for example, a single dose of nobiletin can be 0.01 to 1,000 mg per 1 kg of body weight of an administration target. The above-described administration dose may be 0.15 to 800 mg/kg, 0.5 to 500 mg/kg, 1 to 400 mg/kg, or 1 to 300 mg/kg. The administration dose can be appropriately determined according to disease state, age, sex, body weight, and the like of the administration target.

An administration interval of the pharmaceutical composition according to the present aspect may be appropriately determined according to symptoms, body weight, age, gender, and the like of a patient; a dosage form of the pharmaceutical composition; an administration method; and the like. The administration interval can be set to, for example, every few hours, once a day, once every two or three days, or once a week.

Since the pharmaceutical composition according to the present aspect contains nobiletin having a strong retinal vasodilating action as an active ingredient, it is possible to effectively improve or prevent retinal vascular disturbance. Therefore, a disease related to retinal vascular disturbance can be treated or prevented at an early stage.

Nobiletin contained in the pharmaceutical composition according to the present aspect as an active ingredient is one kind of flavonoid and is a component contained in a large amount in citrus fruits. Therefore, the risk of side effects is low, and the pharmaceutical composition according to the present aspect is suitable for oral and eye drop administration. An effective amount required for improving or preventing retinal circulatory disturbance can reach the retina without side effects.

### OTHER EMBODIMENTS

In an embodiment, the present invention provides a method for treating or preventing retinal circulatory disturbance, the method including administering nobiletin to a subject.

In an embodiment, the present invention provides a method of dilating a retinal blood vessel, the method including administering nobiletin to a subject.

In an embodiment, the present invention provides a method for treating or preventing stenosis or occlusion of a retinal blood vessel, the method including administering nobiletin to a subject.

In an embodiment, the present invention provides a use of nobiletin in manufacturing of a pharmaceutical composition for treating or preventing retinal circulatory disturbance.

In an embodiment, the present invention provides a use of nobiletin in manufacturing of a retinal vasodilator.

In an embodiment, the present invention provides a use of nobiletin in manufacturing of a pharmaceutical composition for treating or preventing stenosis or occlusion of a retinal blood vessel.

In an embodiment, the present invention provides nobiletin for treating or preventing retinal circulatory disturbance.

In an embodiment, the present invention provides nobiletin for dilating a retinal blood vessel.

In an embodiment, the present invention provides a use of nobiletin for treating or preventing stenosis or occlusion of a retinal blood vessel.

In an embodiment, the present invention provides a use of nobiletin for treating or preventing retinal circulatory disturbance.

In an embodiment, the present invention provides a use of nobiletin for dilating a retinal blood vessel.

In an embodiment, the present invention provides a use of nobiletin for treating or preventing stenosis or occlusion of a retinal blood vessel.

### EXAMPLES

Hereinafter, the present invention will be described with reference to Examples, but the present invention is not limited to Examples.

### [Retinal Vasodilation Test Using Porcine Retinal Extraction Artery]

Pigs aged 8 to 12 weeks were euthanized. Immediately after euthanization, the eyeballs were excised. A retinal artery was separated from the excised eyeballs (Nagaoka et al., Invest Ophthalmol Vis Sci. 2007 February; 48(2):825-32.). An anterior eye part and a vitreous body were carefully taken out with an anatomical microscope. A posterior eye part was placed in an ice-cold dissection chamber containing a physiological saline solution (PSS: 145.0 mM NaCl, 4.7 mM KCl, 2.0 mM CaCl₂), 1.17 mM MgSO₄, 1.2 mM NaH₂PO₄, 5.0 mM glucose, 2.0 mM pyruvic acid, 0.02 mM EDTA, and 3.0 mM [3-(N-morpholino) propanesulfonic acid] (MOPS)) to which 1% albumin was added. A single secondary retinal artery (in-situ inner diameter: 90 to 130 μm, length: 0.6 to 1.0 mm) was carefully dissected using a Dumont microdissection forceps (Fine Science Tools, Inc., Foster City, CA) under observation with a stereomicroscope (model SZX12; Olympus, Melville, NY). After carefully removing the remaining nerve tissue and connective tissue, an artery was transferred to a Lucite-made blood vessel chamber into which a PSS-albumin solution equilibrated with air at room temperature was added for cannula insertion. A glass micro pipette (outer diameter of distal end: 30 to 40 μm) filled with the PSS-albumin solution was used to cannulate one end of the artery, and the outside of the artery was firmly tied to a pipette with 11-0 ophthalmic suture (Alcon, Fort Worth, TX). The other end of the blood vessel was cannulated with a second micro pipette and fixed with a suture. After the cannula insertion, the blood vessel and the pipette were transferred to the stage of an inverted microscope (model CKX41, Olympus); and a video camera (Sony DXC-190, Labtek, Campbell, CA), a video micrometer (Cardiovascular Research Institute, Texas A&M System Health Science Center, College Station, TX), and a data collection system (PowerLab, ADInstruments, Colorado Springs, CO) were connected to each other. Thereafter, the inner diameter was continuously measured and recorded during the experiment. The micro pipette was connected to an independent pressure reservoir (a 30 mL glass syringe). The blood vessel was pressurized to a lumen pressure of 55 cm H₂O (40 mmHg) by adjusting the height of the reservoir.

The cannula-inserted arteriole was immersed in PSS at 36° C. to 37° C. to generate a basal tone. After acquiring a stable basal tone of the blood vessel (approximately 30 to 40 minutes), the vasodilating action of nobiletin was evaluated. After the control reaction was terminated, the blood vessel was washed with PSS, and the basal tone was re-generated. Next, nobiletin at each concentration was allowed to act on the blood vessel for 3 to 5 minutes until the diameter of the blood vessel was stabilized.

The blood vessel was relaxed with EDTA (1 mM)-calcium-free PSS, and the maximum diameter was obtained at a lumen pressure of 55 cmH₂O. The change in diameter of the blood vessel in response to nobiletin was standardized by the maximum vasodilation and expressed as a percentage with respect to the maximum vasodilation.

### Example 1

According to the above-described method, a retinal vasodilation test using a porcine retinal extraction artery was performed, and the retinal vasodilating action of nobiletin was evaluated.

As a compound for the positive control, sodium nitropruddide (SNP) was used.

In order to evaluate the role of guanylate synthase in vasodilation by nobiletin, the blood vessel was treated with 1H-[1,2,4]oxadiazolo[4,3,-a]quinoxalin-1-one (ODQ, 0.1 μM), which is a guanylate synthase inhibitor, for 30 minutes. Using the blood vessel, the vasodilating action of nobiletin was evaluated.

### (Results)

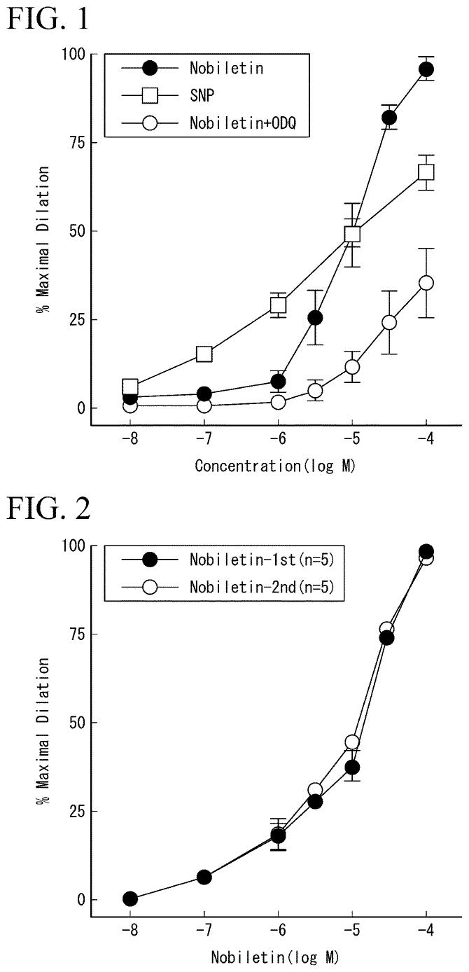

The results are shown in FIG. 1. Nobiletin exhibited a strong retinal vasodilating effect in a dose-dependent manner. The retinal vasodilating effect of nobiletin was higher than that of SNP, which is a standard retinal vasodilating substance. In addition, by treating the blood vessel with ODQ, which is a guanylate cyclase inhibitor, the retinal vasodilating effect of nobiletin was suppressed (Nobiletin+ODQ). From this result, it was suggested that the retinal vasodilating effect of nobiletin was involved in the relaxation of vascular smooth muscle through guanylate cyclase.

### Example 2

According to the above-described method, a retinal vasodilation test (n=5) using a porcine retinal extraction artery was performed twice, and the retinal vasodilating action of nobiletin was evaluated.

The results are shown in FIG. 2. In the two tests, nobiletin exhibited a similar dose-dependent retinal vasodilating effect.

### Example 3

In order to confirm whether the retinal vasodilating action of nobiletin is vascular endothelium-dependent, a retinal artery denuded of endothelium (denudation) was used, which was obtained by perfusing a solution of a surfactant CHAPS into the blood vessel, and a retinal vasodilation test (n=11) using a porcine retinal extraction artery was performed.

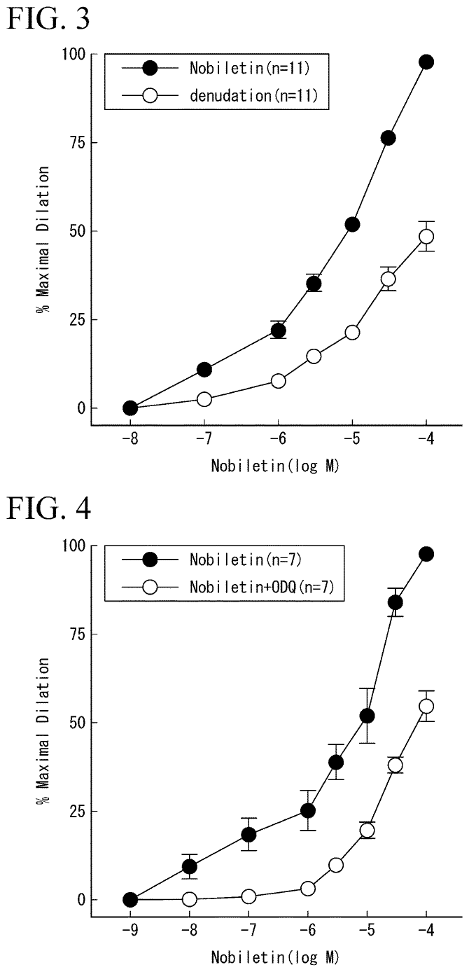

The results are shown in FIG. 3. In the case where the porcine retinal extraction artery from which the vascular endothelium was removed (denudation) was used, the vasodilating effect was suppressed compared with the case in which the porcine retinal artery from which the vascular endothelium was not removed (Nobiretin) was used. From this result, it was suggested that the retinal vasodilating effect of nobiletin was involved in vascular endothelium.

### Example 4

In the same manner as in Example 1, the porcine retinal extraction artery was treated with ODQ, and the role of guanylate cyclase in the retinal vasodilating effect of nobiletin was evaluated (n=7).

The results are shown in FIG. 4. In the same manner as in Example 1, by the addition of ODQ, which is a guanylate cyclase inhibitor, the retinal vasodilating effect of nobiletin was suppressed (Nobiletin+ODQ). From this result, it was strongly suggested that the retinal vasodilating effect of nobiletin was involved in the relaxation of vascular smooth muscle through guanylate cyclase.

### (Comparison Between Nobiletin and Known Medications)

Table 1 shows a comparison of the maximum vasodilation rates of the retinal blood vessel by known medications. The concentration of the solvent used for dissolving the test substance was used in a range which did not affect the vasodilating effect. In the comparison with known medications in which the influence of the solvent was excluded, it was suggested that nobiletin exhibited the most powerful vasodilating action.

TABLE 1

| | | Maximum | |
|---|---|---|---|
| | Concen- | vasodilation | |
| Medication | tration | rate | Reference |
| Nobiletin | 10⁻⁴M | 90% | — |
| Simvastatin | 10⁻⁵M | 50% | Nagaoka et al., |
| | | | Invest Ophthalmol Vis Sci. |
| | | | 2007 February; 48(2): 825-32. |
| Resveratrol | 10⁻⁴M | 60% | Nagaoka et al., |
| | | | Invest Ophthalmol Vis Sci. |
| | | | 2007 September; 48(9): 4232-9. |
| Pioglitazone | 10⁻⁵M | 50% | Omae et al., |
| | | | Invest Ophthalmol Vis Sci. |
| | | | 2011 Aug. 24; 52(9): 6749-56. |
| Fenofibrate | 3 × 10⁻⁵M | 50% | Omae et al., |
| | | | Invest Ophthalmol Vis Sci. |
| | | | 2012 May 14; 53(6): 2880-6. |
| Adiponectin | 8 μg/mL | 50% | Omae et al., |
| | | | Invest Ophthalmol Vis Sci. |
| | | | 2013 Jul. 10; 54(7): 4586-94. |
| Cilostazol | 10⁻⁵M | 40% | Tanano et al., |
| | | | Invest Ophthalmol Vis Sci. |
| | | | 2013 Feb. 19; 54(2): 1443-9. |
| Beraprost | 10⁻⁷M | 70% | Ono et al., |
| | | | Invest Ophthalmol Vis Sci. |
| | | | 2014 Jul. 31; 55(9): 5752-9. |
| Unoprostone | 10⁻⁵M | 30% | Tanano et al., |
| | | | Mol Vis. |
| | | | 2015; 21: 699-705. |

Industrial Applicability

According to the present invention, a novel retinal vasodilator, and a pharmaceutical composition containing the retinal vasodilator are provided.

Although preferred examples of the present invention are described above, the present invention is not limited to these examples. It is possible to add other configurations or to omit, replace, or modify the configurations described herein without departing from the spirit of the present invention. The present invention is not limited by the above description, but only by the scope of the appended claims.

1. A retinal vasodilator comprising: nobiletin.

2. A pharmaceutical composition for treating or preventing retinal circulatory disturbance, the pharmaceutical composition comprising:
the retinal vasodilator according to claim 1; and
a pharmaceutically acceptable carrier.

3. A method of treating or preventing retinal circulatory disturbance, the method comprising administering nobiletin to a subject.
